(19) United States
(12) Patent Application Publication
(10) Pub. No.: US 2003/0099724 A1
(43) Pub. Date: May 29, 2003

(54) Compounds for prevention of diabetic retinopathy

(76) Inventors: Oliver E. Turner, Chesterfield, VA (US); John J. Abbott, Pittsburgh, PA (US); David H. Kinder, Elida, OH (US)

Correspondence Address: PIETRAGALLO, BOSICK & GORDON, ONE OXFORD CENTRE, 38TH FLOOR, 301 GRANT STREET, PITTSBURGH, PA, 15219-6404, US

(21) Appl. No.: 09/998,574

(22) Filed: Nov. 16, 2001

(51) Int. Cl. A61K 33/14; A61K 31/155

(52) U.S. Cl. 424/682; 514/634

(57) ABSTRACT

Compounds for the prevention and retardation of diabetic retinopathy, and the loss of visual acuity and blindness that can be caused by diabetic retinopathy. The compounds may include a magnesium salt, a vasodilator, aminoguanidine, an anti-inflammatory agent, and an antioxidant.

### CROSS-REFERENCE TO RELATED APPLICATIONS

Not applicable.

### FEDERALLY SPONSORED RESEARCH OR DEVELOPMENT

Not applicable.

### BACKGROUND OF THE INVENTION

The present invention pertains to compounds for the prevention and retardation of diabetic retinopathy, and the loss of visual acuity and blindness that can be caused by diabetic retinopathy. More particularly, the present invention pertains to compounds comprised of a magnesium salt, a vasodilator, aminoguanidine, an anti-inflammatory agent, and an antioxidant.

Global statistics indicate that diabetes and diabetic complications affect significant numbers of the population worldwide. The following tables illustrate the significant effects on the population:

TABLE 1
GLOBAL STATISTICS: 1996 POPULATION, DIABETES
30 LARGEST GLOBAL COUNTRIES

| Number | Country | Population 1996 | Diabetic's 5% |
|---|---|---|---|
| 1. | China | 1,179,030,000 | 58,961,150 |
| 2. | India | 874,850,000 | 43,692,500 |
| 3. | United States | 256,430,000 | 12,831,000 |
| 4. | Indonesia | 186,180,000 | 9,309,000 |
| 5. | Brazil | 159,630,000 | 7,981,000 |
| 6. | Russia | 150,500,000 | 7,525,000 |
| 7. | Japan | 124,710,000 | 6,224,500 |
| 8. | Pakistan | 123,492,000 | 6,174,500 |
| 9. | Bangladesh | 120,850,000 | 6,042,500 |
| 10. | Nigeria | 91,700,000 | 4,505,000 |
| 11. | Mexico | 86,170,000 | 4,308,500 |
| 12. | Germany | 80,590,000 | 4,029,500 |
| 13. | Vietnam | 69,650,000 | 3,482,500 |
| 14. | Philippines | 65,500,000 | 3,275,000 |
| 15. | Iran | 60,500,000 | 3,025,000 |
| 16. | Turkey | 58,600,000 | 2,931,000 |
| 17. | Thailand | 58,030,000 | 2,901,500 |
| 18. | United Kingdom | 52,890,000 | 2,894,500 |
| 19. | France | 57,570,000 | 2,878,500 |
| 20. | Egypt | 57,050,000 | 2,852,500 |
| 21. | Italy | 56,550,000 | 2,827,500 |
| 22. | Ukraine | 51,990,000 | 2,594,500 |
| 23. | Ethiopia | 51,715,000 | 2,585,750 |
| 24. | S. Korea | 43,660,000 | 2,183,000 |
| 25. | Miramar | 43,070,000 | 2,153,500 |
| 26. | Zaire | 39,750,000 | 1,987,500 |
| 27. | Spain | 39,156,000 | 1,957,800 |
| 28. | Poland | 38,330,000 | 1,916,500 |
| 29. | Colombia | 34,640,000 | 1,732,000 |
| 30. | S. Africa | 33,017,000 | 1,650,000 |
| TOTALS | 30 Countries | 4,349,808,000 | 217,480,030 |

The population statistics for the 30 largest countries in 1996 are provided by the permission of the Rand McNally Company in Chicago, Ill.

TABLE 2
A. U.S.A.:

| | | |
|---|---|---|
| 1999 Population | 272,000,000 | [Million] |
| 5.9% Diabetes | 16,048,000 | |
| Estimated Type I 10%/Insulin Dependent | 16,048,000 | |
| Estimated Type II 90%/Non Insulin | 14,443,000 | |
| Dependent | | |

B. Global:

| | | |
|---|---|---|
| 1999 Population | 6,200,000,000 | [Billion] |
| 4.35% Diabetes | 267,700,000 | |
| Estimated Type I 10%/Insulin Dependent | 26,970,000 | |
| Estimated Type II 90%/Non Insulin | 242,730,000 | |
| Dependent | | |

Review of medical literature reveals a variable incidence of diabetic retinopathy in group studies. Incidences are reportedly progressive over 5-10-15-20 years from time of clinical diagnosis. In general, a review of the medical literature suggests a 90% incidence of diabetic retinopathy over fifteen years of diabetic treatment. Using these estimates, the following tables illustrate the future significance in the U.S. and worldwide.

TABLE 3
A. U.S.A.
USA-Incidence of Diabetic Retinopathy

| | | | |
|---|---|---|---|
| 1999 | Population | | 272,000,000 |
| 1999 | Diabetics | 5.9% | 16,000,000 |
| 1999 | Retinopathy | 90% | 14,400,000 |
| | | | [15 years] |

Chronological Progression

| | | | |
|---|---|---|---|
| 5 | Years | 33⅓% | 4,795,200 |
| 10 | Years | 66⅔% | 9,590,400 |
| 15 | Years | 100% | 14,400,000 |

U.S.A.-Future Incidence of Diabetic Retinopathy
1999 Through 2020

| | 1999 | 2010 | 2020 |
|---|---|---|---|
| Population | 272,000,000 | 300,000,000 | 324,500,000 |
| Diabetics | 16,000,000 | 17,700,000 | 19,145,500 |
| Retino- | 14,400,000 | 15,930,000 | 17,230,950 |
| pathy | | | |
| 5% | 720,000 | 796,500 | 861,547 |
| Totally | | | |
| Blind | | | |

B. Global
Global-Incidence of Diabetic Retinopathy

| | | | |
|---|---|---|---|
| 1999 | Population | | 6,200,000,000 |
| 1999 | Diabetics | 4.35% | 269,700,000 |
| 1999 | Retinopathy | 90% | 242,730,000 |

Potential Chronological Progression

| | | | |
|---|---|---|---|
| 5 | Years | 33⅓% | 80,829,090 |
| 10 | Years | 66⅔% | 161,658,180 |
| 15 | Years | 100% | 242,730,000 |

Global-Future Incidence of Diabetic Retinopathy
1999 Through 2020

| | 1999 | 2010 | 2020 |
|---|---|---|---|
| Population | 6,200,000,000 | 10,000,000,000 | 12,000,000,000 |
| Diabetics | 272,000,000 | 435,000,000 | 522,000,000 |
| [4.5%] | | | |
| Retino- | 244,800,000 | 391,500,000 | 469,800,000 |
| pathy 90% | | | |
| 5% | 12,240,000 | 19,575,000 | 23,490,000 |
| Totally | | | |
| Blind | | | |

TABLE 4
FUTURE ESTIMATES OF POPULATION AND DIABETICS

| USA | 1999 | 2010 | 2020 |
|---|---|---|---|
| Population | 272,000,000 | 300,000,000 | 324,500,000 |
| Diabetics | 16,000,000 | 17,700,000 | 19,145,500 |

Note: 5.9% Diabetics-Factor 1:17

| Global | 1999 | 2010 | 2020 |
|---|---|---|---|
| Population | 6,200,000,000 | 10,000,000,000 | 12,000,000,000 |
| Diabetics | 269,700,000 | 435,000,000 | 522,000,000 |

Note: 4.35% Diabetics-Factor 1:23

Future estimates for USA based on recent projections of U.S. Census Bureau and present percentages of diabetics in population.

| | | | |
|---|---|---|---|
| USA | Diabetics | 5.9% | of Population |
| Global | Diabetics | 4.35% | of Population |

There are numerous complications associated with the disease diabetes mellitus, which shall hereinafter be referred to as “diabetes.” One such complication is diabetic retinopathy, which is also known as diabetic retinitis or diabetic retinosis. The research efforts of the inventors of the present invention have been directed at prevention of diabetic retinopathy and blindness.

As a result of such research, the inventors recognize that the common retinal pathologic states associated with diabetic retinopathy include non-proliferative and proliferative types of diabetic retinopathy, macular degeneration, retinal detachment, retinitis pigmentosa, vascular changes, emboli, thrombosis, hemorrhage, and hypertensive retinopathy.

The chronological progression of diabetic retinopathy is generally as follows:

| | | |
|---|---|---|
| 0-5 | Years | Early signs of retinopathy-possible early |
| | | changes detected in retinal capillaries, |
| | | thickening or vasospasm. |
| 5-10 | Years | Early to moderate signs-minimal to moderate |
| | | changes in retinal capillaries established by |
| | | ophthalmoscope and fluorescing |
| | | angiographies. |
| 10-15 | Years | Moderate to severe signs-moderate to severe |
| | | changes in retinal capillaries, e.g. micro |
| | | aneurysms, retinal hemorrhage, scarring, etc. |
| 15-20 | Years | Advanced severe signs-advanced changes in |
| | | retinal capillaries, hemorrhage, scarring with |
| | | progressive loss of vision and eventual |
| | | blindness. |

While some clinicians believe that diabetic retinopathy may progress more rapidly in Type I diabetics (insulin dependent patients), e.g., in 5-10 years, diabetic retinopathy occurs both in insulin dependent diabetics and non-insulin dependent diabetics, which suggests that insulin as such is not a main factor in the etiology. Accordingly, an annual eye examination with an ophthalmoscope and retinal angiography is key to the diagnosis of the progression of diabetic retinopathy.

The physiological progression of diabetic retinopathy can be described as “primary systemic vascular pathology,” which occurs in the arteries, arterioles, capillaries, veins and venules of the human body. The primary pathogenesis of diabetic retinopathy occurs in the retinal network of capillaries—a pathologic vasculopathy of the endothelium lining. In brief, the retina is the light sensitive membrane on the back surface of the eye. The optic nerve extends from the brain to the center of the retina and branches out peripherally. The center area of the retina is called the macula where the sharpest visual images are recorded. The retinal artery, arterioles and capillaries circulate out into the retina to provide a rich vascular supply, and especially oxygen, to the retinal membrane. Retinal survival and tissue maintenance depends on rich capillary vascularity and on oxygen availability. During primary systemic vascular pathology, there may be capillary endothelial thickening, capillary endothelial plaques, micro aneurysms in the capillary, with attendant hemorrhage, impaired phagocytosis, or endo-toxic effects on capillary endothelium.

There are multiple patho-physiologic factors that can contribute to the progression of diabetic retinopathy, including physiological, pathological, chemical, immunological, and genetic factors. A few of these factors are discussed below.

Hyperglycemia, whether transient or sustained, can contribute to the progression of diabetic retinopathy. Hyperglycemia creates organic chemical reactions in carbohydrates, proteins, and fats. Abnormal intermediate and end products of such reactions can be metabolized, but some of these products may be toxic agents. The variant of chemical alterations—as endo-toxins—may be toxic to both the retinal capillary endothelium as well to the retinal membrane. Chemical endo-toxins of note include ketose, hexose, and furose. If hyperglycemia is sustained, then the complication of diabetic keto-acidosis may develop over a period of hours or days with ketonemia and ketonuria. Recurrent diabetic ketoacidosis can be particularly damaging.

As illustrated in FIG. 1, the retinal capillary endothelium and the retina are vulnerable to damage caused by microaneurysm. If damage to the retinal arterial capillary system continues, then capillary micro aneurysms can eventually form (steps 1-4 of FIG. 1), and ultimately rupture, leaving “hemo-debris” in the retinal membranes (steps 5-7 of FIG. 1). The list of “hemo debris” constituents includes red blood cells, white blood cells, hemoglobin, platelets, fibrinogen, thromboplastin, plasma proteins such as albumin, globulin fractions, electrolytes, sodium, potassium, chlorides, glucose, toxic end-products of carbohydrates, proteins and fats, ketones (increased by diabetic keto-acidosis), vitamin substances, micro-minerals, cholesterol and lipid fractions, nitric oxide, and lactic acid. If retinal phagocytosis is impaired following such a rupture, then the damage is not healed. This is the basic patho-physiologic mechanism for retinopathy in the diabetic individual, and is sometimes referred to herein as “retinal arterial capillary vasculopathy.” Of particular interest is the organic chemistry of carbohydrate metabolism, and the endotoxins that may be produced by abnormal chemical interactions with intermediaries and end-products of carbohydrate metabolism. For example, there are eight D-glucose isomers known to be intermediaries and end-products in carbohydrate metabolism: D-glucose, D-allose, D-mannose, D-altrose, D-gulase, D-idose, D-galactose, and D-talose. Any of these may have abnormal chemical interactions that result in the creation of endotoxins that can damage the vascular endothelium, in particular, the retinal capillary endothelium, and can affect the pancreatic islet cell production of insulin.

Retinal physiology is dependent on a constant arterial capillary vascular supply of oxygen, minerals, and nutrients. Anoxia can detrimentally affect this supply. Anoxia is a deficit in the oxygen delivery system, and can result in vasospasm, constriction of the retinal capillaries, retinal circulatory blockage, emboli, and thrombosis. Oxygen deprivation of the retina is synonymous with retinal segment death.

In conditions of anoxia and/or the absence of anti-oxidants, nitric oxide and similar super-oxide endo-toxins can contribute to the progression of diabetic retinopathy and can have particularly damaging effects, including retinal capillary vasculopathy. Nitric oxide is a fairly short-lived molecule (with a half life of a few seconds) that is produced from enzymes known as nitric oxide synthases. Its production occurs in human macrophage cells phagocytosing opsonized zymosan. Nitric oxide can cause retinal degeneration and retinal death. The metabolic chemical alterations in carbohydrate chemistry associated with anoxia and restricted oxygen supply, also characterized as anaerobic glycolysis, can produce intermediate and chemical end-products that can be toxic agents, and therefore contribute to diabetic retinopathy.

Progressive axial myopia can also be damaging. It has been proposed that certain compounds such as carbonic acid and lactic acid are important factors in metabolic acidosis, which locally in the eye could create marked scleral edema, and in turn venous congestion at the rear of the ocular bulb. Also, scleral edema, with secondary back pressure on the retinal vein system, could produce a slowdown of the retinal arterial capillary system, and therefore contribute to the development of retinopathy, with aggravated effects in diabetic patients with recurrent endo-toxins. In addition, an excess of carbonated beverages can contribute to scleral edema.

Chemical toxins, bacterial toxins, viral inclusion bodies, bacteriophage and parasites can serve as potential etiologic factors that can contribute to recurrent sustained damage to the retinal arterial capillary system and the retina membrane. Chemical toxins such as cleaning agents, aerosols, paints, and garden chemicals are numerous in the atmosphere and the household. Bacterial toxins such as streptococcal and staphyloccal bacteria occur with childhood and adult infectious diseases. Viral inclusion bodies are known to settle in brain tissue after endemic influenza. Atmospheric chemical endotoxins are numerous, and chiefly consist of methane, carbon dioxide, ammonia, chlorine and chemical solvents. Toxic chemicals such as methyl-tertiary-butyl-ether (MTBE) are also hazardous. In addition, anaerobic methanation in the intestinal tract (colon) with production of methane, and synthesis to methanol is a potential chemical endo-toxin. It appears chemically possible that methane gas in the human colon can be synthesized by virtue of hydrolysis and catalysis to small amounts of methanol which may be absorbed into the blood stream, and become a chemical endotoxin to both the pancreas and the retina—an unusual, but nonetheless possible, etiologic consideration for retinopathy. It cannot be assessed at this time whether methane—to methanol—is related to diabetic or non-diabetic individuals with impaired carbohydrate metabolism.

Hypertension is important as a transient or sustained insult that can contribute to the progression of diabetic retinopathy. For example, hypertension can cause capillary vaso-constriction of retinal vessels. Furthermore, a sudden surge of blood pressure in a state of tension, anger or rage could cause rupture of retinal capillary micro-aneurysms with retinal hemorrhage and loss of visual acuity. Repeated capillary endothelial changes can lead to eventual micro-aneurysms that can rupture with stress and hypertension, and with blood extravasation into the retina to alter visual acuity.

Atherosclerosis of the retinal arterial system can occur progressively in a diabetic patient and contribute to the progression of diabetic retinopathy. Contributing factors to this vasculopathy are hypercholesterolemia and hypertri-glyceridemia.

Progressive patho-physiologic changes in the retinal capillary endothelium, due to any of the factors previously discussed, lead to degenerative weakening of the endothelium with eventual formation of retinal capillary micro-aneurysms. With stress, the micro-aneurysms rupture, and spill the capillary blood constituents into the retinal membrane. This is the first stage of micro-hemorrhage in the retina of diabetics. The next physiologic consequence is the attempt of repair. This repair process is called retinal phagocytosis. By definition, retinal phagocytosis is the mechanism of ingestion or digestion of “hemo-debris.” Monocytes engulf the foreign particles extravasated in the retinal membrane. The repair process may be normal or impaired. Multiple ruptures of retinal capillary micro-aneurysms lead to progressive stages of diabetic retinopathy and loss of visual acuity. Thus, the repair process, retinal phagocytosis, is an important consideration in a study of the progression of diabetic retinopathy. The present invention focuses on retinal capillaries as the heart of the development of diabetic retinopathy. Of primary interest is the sensitivity and involvement of the endothelium of retinal capillaries and retinal venules.

Additional factors that can contribute to the progression of diabetic complications such as diabetic retinopathy include long term dietary imbalances, vitamin and mineral deficiencies, genetic defects in chemical metabolism, and anaerobic glycolysis.

General considerations in preventative therapy for diabetes and its complications such as diabetic retinopathy include early diagnosis, awareness of medical, nursing, and public health professionals, long-term monitoring of progressive stages of diabetic retinopathy by ophthalmologists, calorie controlled diabetic diets, e.g., 1800 calories, moderation in carbonated beverages, control of weight problems, avoidance of toxic chemicals, clean drinking water, careful personal hygiene, and daily vitamin and mineral supplements.

The importance of compositions comprising various vitamins and minerals in the treatment of complications of diabetes is known in the art as represented by U.S. Pat. Nos. 5,871,769 to Fleming et al.; 6,103,756 to Gorsek; 5,849,338 to Richardson et al.; 6,042,849 to Richardson et al., 5,962,030 to Fine; and 5,128,360 to Cerami et al. Despite these compositions, however, complications of diabetes that result in a loss of visual acuity and blindness persist, and thus a need remains for alternative compositions. The present invention comprises compounds to prevent diabetic retinopathy and retard its progression. Such compounds preferably include a combination of five components administered in a daily dosage for the life of the diabetic, to retard and prevent loss of visual acuity and blindness in long term diabetic treatment.

### BRIEF SUMMARY OF THE INVENTION

According to the present invention, orally ingested compounds are provided for the prevention of diabetic retinopathy and the retardation of its progression. The compounds of the present invention can be used as a long-term approach, over the clinical course of diabetic patients, tailored to prevent and retard the retinopathic process, by preventing the following: transient acidosis, transient vasospasm of retinal capillary function, aberrant chemical endotoxins in carbohydrates, protein, and fat chemisty, hyperglycemia and wide variations in daily glucose levels, and other etiologic factors in retinopathy.

According to preferred embodiments of the present invention, the compounds comprise therapeutically effective amounts of a source of magnesium, a vasodilator, aminoguanidine, an anti-inflammatory agent, and an antioxidant. The compounds are administered as a tablet or liquid, daily, or as required to be therapeutically effective in humans. A preferred embodiment of the present invention is a compound comprising magnesium carbonate, amlodipine besylate, amino guanidine, acetyl salicylic acid (aspirin), and ascorbic acid (Vitamin C).

### BRIEF DESCRIPTION OF THE DRAWINGS

FIG. 1 is a schematic diagram of how microaneurysms form and burst, thereby damaging the retinal capillary endothelium and the retina.

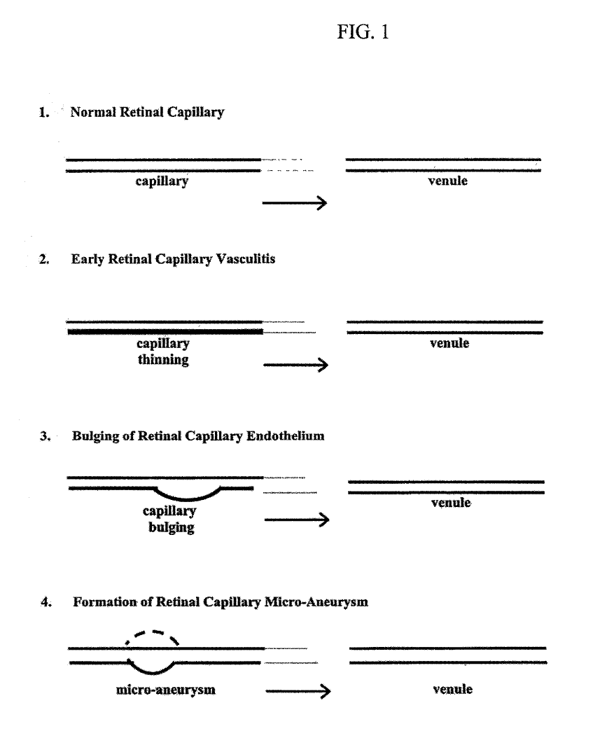

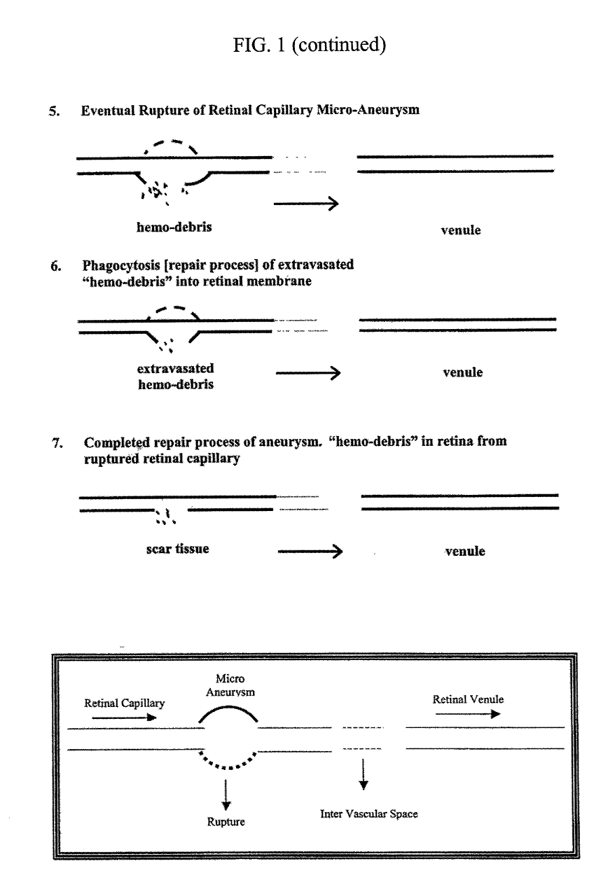

### DETAILED DESCRIPTION OF THE INVENTION

The compounds of the present invention for the prevention of diabetic retinopathy and the retardation of its progression comprise effective amounts of at least two of the following: a source of magnesium, a vasodilator, aminoguanidine, an anti-inflammatory agent, and an antioxidant.

Magnesium has an important role in the following: as an essential micro-mineral, protection of the retinal capillary endothelium, protection of the arterial endothelial lining, as a catalytic agent in cellular metabolism, bone structure, formation of cyclic AMP, ion movements across cell membranes, carbohydrate metabolism and protein synthesis. Important enzymes such as phosphatases are magnesium-activated and magnesium-dependent. Further, magnesium is required for thiamine pyro-phosphate co-factor activity, and appears to stabilize the macro-molecular structures RNA and DNA. Magnesium may also be important to the regulation of blood pressure, may act as a physiologic calcium channel blocker, and low levels of magnesium if in the blood may play a part in insulin resistance. In addition, extra-cellular magnesium is critical to maintaining nerve impulses and transmitting impulses across neuromuscular junctions.

According to preferred embodiments of the present invention, the source of magnesium in the compound is one or more magnesium salts selected from magnesium carbonate, magnesium citrate, magnesium chloride, magnesium fumarate, magnesium malate, magnesium glutorate, magnesium lactate, magnesium stearate, magnesium acetate, magnesium ascorbate, magnesium taurate, magnesium orotate, magnesium diglycinate, magnesium oxide, and magnesium succinate. The compounds of the present invention preferably comprise magnesium carbonate, although as noted above, and as will be recognized by those of ordinary skill in the art, magesium carbonate is but one of a variety of magnesium salts suitable for use in the compounds of the present invention. The compounds of the present invention seek to overcome a deficiency in magnesium ion that may be present in humans suffering from diabetic retinopathy.

According to the compounds of the present invention, magnesium is administered to a human in a therapeutically effective amount, which is that amount that increases the human's magnesium level, that amount that reduces recurrent acidosis, or that amount which retards the progression of diabetic retinopathy in the human. While magnesium carbonate has been associated with gastrointestinal side effects, it is unlikely that side effects such as diarrhea would occur at the dose contemplated by the invention. A preferred dose of magnesium according to the compounds of the present invention is about 40 to about 400 mg per day. More preferably, the dose of magnesium is about 40 mg for an infant (under 3 years), about 200 mg for a child (3-16 years), and about 400 mg for an adult.

According to preferred embodiments, the compounds of the present invention comprise a vasodilator. A vasodilator can be used to counter the vasoconstrictive effect of nitric oxide within a human body. Calcium channel blockers and angiotensin converting enzyme (ACE) inhibitors are suitable vasodilators for use in the compounds of the present invention. ACE inhibitors provide the additional benefit of improving renal function. Suitable ACE inhibitors for use in the compounds of the present invention include captopril (marketed as Capoten® by Bristol Myers Squibb), enalapril (marketed as Vasotec® by Merck & Co.), lisinopril (marketed as Zestril® by Astra Zeneca), fosinopril (marketed as Monopril® by Bristol Meyers Squibb), benazapril (marketed as Lotensin® by Novartis Pharmaceuticals), and ramipril (marketed as Altace® by King Pharmaceuticals). The ACE inhibitors inhibit the formation of angiotensin II, a highly potent vasoconstrictor as well as the secretion of aldosterone.

According to other preferred embodiments, the compounds of the present invention may include metalloproteinase inhibitors, which are dual inhibitors of ACE and neutral endopeptidase (NEP).

According to still another preferred embodiment, compounds of the present invention comprise amlodipine besylate, the besylate salt of amlodipine (brand name Norvasc® by Pfizer Pharmaceutical). Amlodipine besylate is a white crystalline powder that is slightly soluble in water and sparingly soluble in alcohol. There are no known side effects from daily long term administration of small dosages of amlodipine besylate. Possible side effects include swelling of the ankles, mild dizziness, a pounding heartbeat, lightheadedness, flushing, headache, and tiredness.

As a long-acting calcium channel blocker (CCB), amlodipine besylate is useful in treating angina and hypertension. Amlodipine besylate acts as a mild anti-hypertensive agent with secondary vasodilation effect. It may promote stability of retinal capillary function, help control vasospasm in retinal arterioles and capillary circulation, and preserve constant oxygen supply. Amlodipine besylate affects the movement of calcium into the cells of the heart and blood vessels. Amlodipine besylate also relaxes blood vessels (control of vasospasm) and thus increases the blood supply and oxygen to the heart, and similarly to the retinal circulation.

Other suitable CCBs for use in the compounds of the present invention include: nifedipine (marketed as Procardia® by Pfizer) nicardipine (marketed as Cardene® by Roche Pharmaceuticals), felodipine (marketed as Plendil® by Astra Zeneca), nimodipine (marketed as Nimotop® by Bayer Pharmaceuticals), nisoldipine (marketed as Nisocor® by Bayer Pharmaceuticals), isradipine (marketed as Dynacirc® by Novartis Pharmaceuticals). Still other widely available suitable agents for the compounds of the present invention that inhibit calcium ion influx include verapamil (marketed as Calan® by Searle) and diltiazem (marketed as Cardizem® by Hoescht Marion Roussell).

Most preferably, the compounds of the present invention comprise at least one of amlodipine besylate and nicardipine for the retardation of the progression of diabetic retinopathy. Amlodipine besylate and nicardipine are preferred since they contain an additional amino group compared to the other CCB's, and are more lipophilic, which allows for greater tissue penetration. Amlodipine besylate relaxes blood vessels, thereby controlling vasopasm and increasing the blood and oxygen supply to the heart and to the retinal circulation of the eye.

According to the compounds of the invention, the vasodilator is administered to humans in a therapeutically effective amount, which is that amount that either counters vasoconstriction or that retards the progression of diabetic retinopathy. Preferably, the vasodilator is amlodipine besylate. A preferred dose of amlodipine besylate according to the compounds of the present invention is about 0.4 mg to about 10 mg per day. More preferably, amlodipine besylate is not administered to an infant (under 3 years), but is administered at about 0.5 to about 2.5 mg for a child (3-16 years), and about 2.5 to about 10 mg for an adult (16+years).

According to another preferred embodiment, the vasodilator is nifedipine (marketed as Adalat® by Bayer, Procardia® by Pfizer, or generically by Geneva, Goldine, Moore, Major, Rugby, and others). A preferred dose of nifedipine according to the compounds of the present invention is about 1 mg to about 20 mg per day. More preferably, nifedipine is not administered to an infant (under 3 years), but is administered at about 1 to about 1.5 mg for a child (3-16 years), and about 5 to about 20 mg for an adult (16+ years).

According to another preferred embodiment, the vasodilator is the ACE inhibitor enalapril (marketed as Vasotec® by Merck & Co.). A preferred dose of enalapril according to the compounds of the present invention is about 0.1 mg to about 20 mg per day. More preferably, the dose of enalapril is less than 2.5 mg per day for an infant (under 3 years), about 2.5 to about 5.0 mg per day for a child (3-16 years), and about 2.5 to about 20 mg per day for an adult (16+ years).

According to another preferred embodiment, lisinopril (Zestril® by Astra Zeneca) is the ACE inhibitor. A preferred dose of lisinopril according to the compounds of the present invention is about 5-40 mg per day. Lisinopril is not recommended for an infant (under 3 years). More preferably, the dose of lisinopril is about 5-10 mg per day for a child (3-16 years), and about 20-40 mg per day for an adult (16+ years).

According to other preferred embodiments of the present invention, other suitable vasodilators, ACE inhibitors, and CCBs are used in doses that cause antihypertensive effects.

According to a preferred embodiment, the compounds of the present invention comprise amino guanidine. Amino guanidine may prevent leukocyte dysfunction. Amino guanidine inhibits reactive oxygen formation, for example, lipid perioxidation, and oxidant induced apoptosis. Amino guanidine is effective against plasma nitric oxide, is an iso-form selective mechanism based inactivator of nitric oxide synthase, and may be effective in diabetic neuropathy. In rat models, amino guanidine retards the progression of diabetic retinopathy. Its pharmacological kinetics are effective in mice, and its combined effects with dexamethasone in rats are effective against chemical endotoxins. Amino guanidine appears to inhibit nitric oxide synthase, prevent damage to pancreatic islet beta cells, and prevent damage to retinal arterioles, capillaries, and the retinal membrane. In addition, amino guanidine may neutralize the effects of bacterial endo-toxins, neutralize the effect of nitric acid on the retina, renal glomeruli, and pancreatic beta islet cells, and may stimulate retinal phagocytosis of extravasated blood in the retinal membrane resulting from a ruptured micro-aneurysm.

According to the compounds of the invention, amino guanidine is administered to humans in an amount effective to inhibit nitric oxide synthase. A preferred dose of amino guanidine is about 10 mg-50 mg per day. More preferably, a preferred dose is about 10 mg per day for an infant (1-3 years), about 30 mg per day for a child (3-16 years), and about 50 mg per day for an adult (16+ years).

According to a preferred embodiment, the compounds of the present invention comprise an anti-inflammatory agent, preferably, acetyl salicylic acid (aspirin). Aspirin blocks the synthesis of thromboxane, retards vascular thrombosis in veins and venules by preventing platelet aggregation, has a “slickening” effect on the vascular endothelium, arterioles, capillaries, and retinal membrane, and may retard “clogging” of retinal arterioles and capillaries.

According to the compounds of the invention, the anti-inflammatory agent, preferably aspirin, is administered to humans in an amount effective to prevent platelet aggregation. A preferred dose of aspirin is 20 to 650 mg per day. More preferably, the dose of aspirin is about 20 to about 84 mg per day for an infant (0-3 years), about 80 to about 325 mg per day for a child (3-16 years), and about 80 to about 650 mg per day for an adult (16+ years).

Those of ordinary skill in the art will recognize that other non-steroidal anti-inflammatory agents (NSAIDS) suitable for use in the compounds of the present invention, include the following, listed by generic name: diclofenac, difluisal, etodolac, fenoprofen, flurbiprofen, ibuprofen, indomethacin, ketoprofen, meclofenamate, nabumetone, naproxen, choline salicylate, oxaprozin, piroxicam, sulindac, and tolmetin.

According to another preferred embodiment of the present invention, the compounnd includes ibuprofen as the anti-inflammatory agent. A preferred dose of ibuprofen is about 50 to about 400 mg per day. More preferably, the dose of ibuprofen is about 50 to about 100 mg per day for an infant (0-3 years), about 50 to about 200 mg per day for a child (3-16 years), and about 200 to about 400 mg per day for an adult (16+ years).

According to other preferred embodiments of the present invention, the compounds include naproxen as the anti-inflammatory agent. A preferred dose of naproxen is about 10 to about 200 mg per day. More preferably, the dose of naproxen is about 10 to about 50 mg per day for an infant (0-3 years), about 50 to about 100 mg per day for a child (3-16 years), and about 100 to about 200 mg per day for an adult (16+ years).

The compounds of the present invention preferably comprise an anti-oxidant. The anti-oxidant helps to prevent retinal capillary fragility, retinal capillary hemorrhage, glucose intolerance, and insulin resistance. A preferred anti-oxidant is Vitamin C.

According to the compounds of the invention, the anti-oxidant is administered to humans in a therapeutically effective amount, which is that amount that retards the progression of diabetic retinopathy. The preferred dose of Vitamin C is about 10 to about 250 mg per day. More preferably, the dose of Vitamin C is about 10 to about 50 mg per day for an infant (under 3 years), about 25 to about 100 mg per day for a child (3-16 years), and about 50 to about 250 mg per day for an adult (16+ years).

In addition to Vitamin C, other suitable anti-oxidants for use in the compounds of the present invention include: lutein, bilberry extract and natural vitamin E. According to another preferred embodiment of the present invention, the antioxidant is Vitamin E. A preferred dose of Vitamin E is about 25 to about 800 IU per day. More preferably, the dose of Vitamin E is 25 to about 100 IU per day for an infant (under 3 years), about 100 to about 400 IU per day for a child (3-16 years), and about 100 to about 800 IU per day for an adult (16+ years).

In accordance with a preferred embodiment, the compounds of the present invention are provided in a capsule dosage form. More preferably, unwanted interaction between the aspirin and the other ingredients in the capsule is substantially prevented by providing a protective coating around the aspirin. For example, a protective coating may be provided around the aspirin that minimizes or prevents deleterious reactions with the other vitamin and mineral ingredients. Methods of coating compounds such as aspirin are well-known to those of ordinary skill in the art, as illustrated by U.S. Pat. No. 6,274,170, the entire disclosure of which is incorporated herein by reference.

Preferably, the protective coating comprises at least one layer of wax, shellac, hydroxypropylmethylcellulose phthalate, polyvinyl acetate phthalate and/or cellulose acetate phthalate. In a preferred embodiment, the protective coating comprises an enteric coating which may be applied to tablet formulations or to drug particles or granules used in the subsequent fabrication of capsules. The coating systems can be either aqueous based or organic solvent based to resist breakdown in the low pH environment of the stomach.

Preferably, the capsule dosage form of the present invention comprises aspirin, coated or uncoated, combined with any or all of amino guanidine, a vasodilator, a magnesium salt, and an anti-oxidant. Methods for filling capsules are known to those of ordinary skill in the art.

In addition to the preferred capsule dosage form, the compounds of the present invention could be made in other dosage forms, including but not limited to a bilayered tablet, a sustained release capsule, a transdermal patch, a liquid, and a colloidal suspension. Methods for making each such dosage form are known to those of ordinary skill in the art.

A preferred compound according to the present invention comprises at least a calcium channel blocker, amino guanidine, magnesium carbonate, and aspirin. However, those of ordinary skill in the art will recognize that other compounds, comprised of more or less compounds, can have therapeutic effects. Furthermore, although illustrative embodiments of the invention have been described, a wide range of modification, change, and substitution is intended in the disclosure herein, and in some instances, some features of the present invention may be employed without a corresponding use of the other features. Accordingly, it is appropriate that the appended claims be construed broadly and in a manner consistent with the scope of the invention.

What is claimed is:

1. An orally ingested compound for preventing or retarding retinal pathologic states associated with diabetic retinopathy comprising:
therapeutically effective amounts of at least two members selected from the group consisting of a source of magnesium, a vasodilator, amino guanadine, an anti-inflammatory agent, and an antioxidant.

2. The compound of claim 1 wherein said compound comprises amino guanidine and a source of magnesium.

3. The compound of claim 1 wherein said compound comprises amino guanidine and an anti-inflammatory agent.

4. The compound of claim 1 wherein said compound comprises amino guanidine and an anti-oxidant.

5. The compound of claim 1 wherein said compound comprises amino guanidine and a vasodilator.

6. The compound of claim 5 further comprising a source of magnesium.

7. The compound of claim 5 further comprising an anti-inflammatory agent.

8. The compound of claim 5 further comprising an anti-oxidant.

9. The compound of claim 1 wherein said compound comprises a vasodilator and a source of magnesium.

10. The compound of claim 1 wherein said compound comprises a vasodilator and an anti-inflammatory agent.

11. The compound of claim 1 wherein said compound comprises a vasodilator and an anti-oxidant.

12. The compound of claim 1 comprising at least three members selected from said group.

13. The compound of claim 1 comprising at least four members selected from said group.

14. The compound of claim 1 comprising a source of magnesium, a vasodilator, amino guanadine, an anti-inflammatory agent, and an antioxidant.

15. The compound of claim 1 wherein said source of magnesium is selected from the group consisting of magnesium carbonate, magnesium citrate, magnesium chloride, magnesium fumarate, magnesium malate, magnesium glutorate, magnesium lactate, magnesium stearate, magnesium acetate, magnesium ascorbate, magnesium taurate, magnesium orotate, magnesium diglycinate, magnesium oxide, and magnesium succinate.

16. The compound of claim 1 wherein said source of magnesium comprises magnesium carbonate.

17. The compound of claim 1 wherein said vasodilator is selected from the group consisting of calcium channel blockers, angiotensin converting enzyme inhibitors, and metalloproteinase inhibitors.

18. The compound of claim 1 wherein said vasodilator is a calcium channel blocker selected from the group consisting of amlodipine besylate, nifedipine, nicardipine, felodipine, nimodipine, nisoldipine, isradipine, verapamil, and diltiazem.

19. The compound of claim 1 wherein said vasodilator is an angiotensin converting enzyme inhibitor selected from the group consisting of captopril, enalapril, lisinopril, fosinopril, benazapril, and ramipril.

20. The compound of claim 1 wherein said vasodilator comprises amlodipine besylate.

21. The compound of claim 1 wherein said anti-inflammatory agent is selected from the group consisting of acetal salicylic acid, diclofenac, difluisal, etodolac, fenoprofen, flurbiprofen, ibuprofen, indomethacin, ketoprofen, meclofenamate, nabumetone, naproxen, choline salicylate, oxaprozin, piroxicam, sulindac, and tolmetin.

22. The compound of claim 1 wherein said anti-inflammatory agent comprises acetyl salicylic acid.

23. The compound of claim 1 wherein said anti-oxidant is selected from the group consisting of Vitamin C, Vitamin E, lutein and bilberry extract.

24. The compound of claim 1 wherein said anti-oxidant comprises Vitamin C.

25. The compound of claim 14 comprising about 40 to about 400 mg of magnesium carbonate, about 0.4 to about 10 mg of amlodipine besylate, about 10 to about 50 mg of aminoguanadine, about 20 to about 650 mg of acetyl salicylic acid, and about 10 to about 250 mg of Vitamin C.

26. The compound of claim 16 comprising about 40 to about 400 mg of magnesium carbonate.

27. The compound of claim 20 comprising about 0.4 to about 10 mg of amlodipine besylate.

28. The compound of claim 1 wherein said compound comprises about 10 to about 50 mg of aminoguanidine.

29. The compound of claim 22 comprising about 20 to about 650 mg of acetyl salicylic acid.

30. The compound of claim 24 comprising about 10 to about 250 mg of Vitamin C.

31. A method for preventing or retarding retinal pathologic states associated with diabetic retinopathy in a mammal comprising orally administering to said mammal a compound comprising:
therapeutically effective amounts of at least two members selected from the group consisting of a source of magnesium, a vasodilator, amino guanadine, an anti-inflammatory agent, and an antioxidant.

32. The method of claim 31 wherein said compound comprises amino guanidine and a source of magnesium.

33. The method of claim 31 wherein said compound comprises amino guanidine and an anti-inflammatory agent.

34. The method of claim 31 wherein said compound comprises amino guanidine and an anti-oxidant.

35. The method of claim 31 wherein said compound comprises amino guanidine and a vasodilator.

36. The method of claim 35 wherein said compound further comprises a source of magnesium.

37. The method of claim 35 wherein said compound further comprises an anti-inflammatory agent.

38. The method of claim 35 further comprising an anti-oxidant.

39. The method of claim 31 wherein said compound comprises a vasodilator and a source of magnesium.

40. The method of claim 31 wherein said compound comprises a vasodilator and an anti-inflammatory agent.

41. The method of claim 31 wherein said compound comprises a vasodilator and an anti-oxidant.

42. The method of claim 31 wherein said compound comprises at least three members selected from said group.

43. The method of claim 31 wherein said compound comprises at least four members selected from said group.

44. The method of claim 31 wherein said compound comprises a source of magnesium, a vasodilator, amino guanadine, an anti-inflammatory agent, and an antioxidant.

45. The method of claim 31 wherein said source of magnesium is selected from the group consisting of magnesium carbonate, magnesium citrate, magnesium chloride, magnesium fumarate, magnesium malate, magnesium glutorate, magnesium lactate, magnesium stearate, magnesium acetate, magnesium ascorbate, magnesium taurate, magnesium orotate, magnesium diglycinate, magnesium oxide, and magnesium succinate.

46. The method of claim 31 wherein said source of magnesium comprises magnesium carbonate.

47. The method of claim 31 wherein said vasodilator is selected from the group consisting of calcium channel blockers, angiotensin converting enzyme inhibitors, and metalloproteinase inhibitors.

48. The method of claim 31 wherein said vasodilator is a calcium channel blocker selected from the group consisting of amlodipine besylate, nifedipine, nicardipine, felodipine, nimodipine, nisoldipine, isradipine, verapamil, and diltiazem.

49. The method of claim 31 wherein said vasodilator is an angiotensin converting enzyme inhibitor selected from the group consisting of captopril, enalapril, lisinopril, fosinopril, benazapril, and ramipril.

50. The method of claim 31 wherein said vasodilator comprises amlodipine besylate.

51. The method of claim 31 wherein said anti-inflammatory agent is selected from the group consisting of acetal salicylic acid, diclofenac, difluisal, etodolac, fenoprofen, flurbiprofen, ibuprofen, indomethacin, ketoprofen, meclofenamate, nabumetone, naproxen, choline salicylate, oxaprozin, piroxicam, sulindac, and tolmetin.

52. The method of claim 31 wherein said anti-inflammatory agent comprises acetyl salicylic acid.

53. The method of claim 31 wherein said anti-oxidant is selected from the group consisting of Vitamin C, Vitamin E, lutein and bilberry extract.

54. The method of claim 31 wherein said anti-oxidant comprises Vitamin C.

55. The method of claim 44 wherein said compound comprises about 40 to about 400 mg of magnesium carbonate, about 0.4 to about 10 mg of amlodipine besylate, about 10 to about 50 mg of aminoguanadine, about 20 to about 650 mg of acetyl salicylic acid, and about 10 to about 250 mg of Vitamin C.

56. The method of claim 46 wherein said compound comprises about 40 to about 400 mg of magnesium carbonate.

57. The method of claim 50 wherein said compound comprises about 0.4 to about 10 mg of amlodipine besylate.

58. The method of claim 31 wherein said compound comprises about 10 to about 50 mg of aminoguanidine.

59. The method of claim 52 wherein said compound comprises about 20 to about 650 mg of acetyl salicylic acid.

60. The method of claim 54 wherein said compound comprises about 10 to about 250 mg of Vitamin C.
